(19) United States
(12) Patent Application Publication
(10) Pub. No.: US 2026/0028888 A1
(43) Pub. Date: Jan. 29, 2026

(54) Intelligent Cuttings Sampler

(71) Applicant: GEOLOG S.r.l., San Giuliano Milanese (MI) (IT)

(72) Inventor: Antonio Calleri, San Giuliano Milanese (IT)

(21) Appl. No.: 19/284,291

(22) Filed: Jul. 29, 2025

(30) Foreign Application Priority Data
Jul. 29, 2024 (IT) 102024000017593

(51) Int. Cl. E21B 21/06; E21B 21/08; E21B 45/00; E21B 49/00; G01N 1/40; G01N 33/28; G06V 10/764; G06V 10/98; G06V 20/50

(52) U.S. Cl. CPC E21B 21/066; E21B 21/08; E21B 45/00; E21B 49/005; G01N 1/40; G01N 33/2823; G06V 10/764; G06V 10/98; G06V 20/50

(57) ABSTRACT

A cuttings sampler for testing of cuttings from a drilling operation includes a sample extractor which extracts a sample of drilling mud from a flow line. The sample extractor includes a probe located inside of the flow line and having a plurality of openings to allow for the passage of drilling mud containing cuttings, and a variable flow mud pump for extracting a drilling mud sample from the mud flow line. The cuttings sampler further includes: a cleaning system that performs a cleaning action to remove fluid in the drilling mud sample resulting in a cutting sample; an analysis system for performing an initial analysis on the cutting sample to classify a type of rock contained in the cutting sample; a packaging system for accepting and placing the cutting sample in a storage container; and a main controller, configured to control at least the variable flow mud pump.

### CROSS-REFERENCE TO RELATED APPLICATIONS

This application claim priority to Italian Application No. 102024000017593, filed Jul. 29, 2024, which is incorporated herein by specific reference.

### BACKGROUND OF THE DISCLOSURE

Drilling operations require a staggering amount of information to make crucial decisions based on the analysis of a well and its economic potential. The information related to the rock formations into which the drilling operation is conducted/performed is of special importance and falls under the domain of mud logging. During a drilling operation, the cuttings generated by the drill bit are often the only physical lithological data that can be recovered from a well. Proper sampling and analysis of the cuttings then represents a major technical aspect of a drilling process.

The mud logging process comprises the steps of collecting, cleaning, analyzing, and archiving samples of the drilled rock formations as a function of the depth reached by the drill bit. A mud logger is a person responsible for performing those duties. The quality of the cuttings samples relies on the capability of the mud logger to avoid cavings, sample at various frequencies, properly clean the samples from the drilling mud, and preserve the representativeness of the sampled rock formation.

A human operator tasked with performing an analysis of this type has difficulty performing quick and repeated analyses of the cuttings coming from a drilling mud. The speed of sampling may vary depending upon the types and content of the drilling mud itself. A single operator may be expected to collect a sample every 10 minutes on average. In some cases, additional personnel may be employed; their added cost may be prohibitive for certain applications and affect the collection of samples.

It is then in a company's interest to improve the sampling capabilities at a wellsite, without resorting to additional personnel for an improved analysis.

The present invention is then an intelligent cuttings sampler which enables automation of the mud-logging workflow. The addition of intelligent and controlled elements to carry-out/perform the mud-logging process steps increases the precision and safety of the mud-logging process while decreasing the costs associated with performing the sampling.

### SUMMARY OF THE DISCLOSURE

The present invention provides an intelligent cuttings sampler to avoid the limitations mentioned above.

In particular, the present invention provides an intelligent cuttings sampler for the retrieval and testing of drilling mud cuttings from a drilling operation.

Preferably, the intelligent cuttings sampler comprises a sample extractor.

Preferably, the intelligent cuttings sampler comprises a cleaning system.

Preferably, the intelligent cuttings sampler comprises an analysis system.

Preferably, the intelligent cuttings sampler comprises a packaging system.

Preferably, the intelligent cuttings sampler comprises a main controller.

Preferably, the sample extractor is configured to extract a sample of drilling mud from a mud return system.

Preferably, the sample extractor comprises a probe.

Preferably, the probe is located inside of the mud return system.

Preferably, the probe has a plurality of openings.

Preferably, the plurality of openings are sized to allow for the passage of drilling mud containing cuttings.

Preferably, the sample extractor comprises a variable flow mud pump.

Preferably, the cleaning system is configured to accept the drilling mud sample provided by the sample extractor.

Preferably, the cleaning system performs a cleaning action to remove fluid in the drilling mud sample.

Preferably, the analysis system is configured to perform an initial analysis on the cutting sample.

Preferably, the analysis system is configured to classify a type of rock contained in the cutting sample.

Preferably, the packaging system is configured to receive the cutting sample.

Preferably, the packaging system is configured to place the sample in a storage container.

Preferably, the main controller is configured to control at least the variable flow mud pump.

Preferably, the control is a function of a rate of penetration of the drilling operation.

Preferably, the rate of penetration is based on an estimate of an acceptable amount of cuttings contained in a drilling mud sample.

Preferably, the probe includes a cleaner.

Preferably, the cleaner is configured to maintain a plurality of probe openings free of obstructions.

Preferably, the plurality of openings have a dimension of between 3 mm and 4 mm.

Preferably, said cleaning system comprises an initial shale-shaker.

Preferably, said initial shale-shaker is configured to accept a drilling mud sample from said sample extractor and separate cuttings from the drilling mud.

Preferably, the intelligent cuttings sampler further comprises a fume hood.

Preferably, the intelligent cuttings sampler further comprises a gas sensor.

Preferably, the gas sensor is configured to send information pertaining to a quantity or concentration of a hazardous gas.

Preferably, the initial shale-shaker is covered by said fume hood.

Preferably, the cleaning system further comprises a washing station.

Preferably, said washing station is configured to perform a first washing action.

Preferably, said first washing action is performed with water or solvent selected based on a type of drilling mud.

Preferably, said washing station in configured to perform a second washing action.

Preferably, said second washing action is performed with a detergent.

Preferably, said washing station is configured to perform a third washing action.

Preferably, said third washing action is performed with water.

Preferably, the cleaning system further comprises a drying station.

Preferably, said drying station is configured to perform a drying operation.

Preferably, said drying operation is performed using a heating element.

Preferably, said drying operation is performed using a vibration element.

Preferably, the cleaning system further comprises an imaging system.

Preferably, said imaging system is located between said washing and drying station.

Preferably, the imaging system is configured to analyze a cleanliness level of the cutting sample.

Preferably, said imaging system is configured to analyze the cleanliness level of said cutting sample after said first washing action.

Preferably, said imaging system is configured to analyze the cleanliness level of said cutting sample after said second washing action.

Preferably, the analysis system comprises a photography device capable of taking photographs in ultra-violet and visible light.

Preferably, the analysis system is configured to classify the rock contained in the cutting sample based on the output of a trained machine learning algorithm.

Preferably, the packaging system is configured to separate a subset of said cutting sample for additional analysis.

Preferably, the intelligent cuttings sampler further comprises a first conveyor belt.

Preferably, the first conveyor belt is provided with a plurality of mesh containers.

Preferably, said mesh containers are configured to accept said drilling mud sample and enable said cutting sample for additional analysis.

Preferably, the intelligent cuttings sampler further comprises a transport device.

Preferably, the transport device contains a plurality of sample containers.

Preferably, said plurality of sample containers are configured to transfer the cutting sample between the cleaning system, analysis system, and packaging system and allow for compact layout of said cleaning, analysis, and packaging systems.

Preferably, the intelligent cuttings sampler further comprises a second conveyor belt.

Preferably, the second conveyor belt is configured to receive said cuttings sample from the first conveyor belt and bring said cuttings sample to said transport device.

Preferably, the intelligent cuttings sampler comprises a vessel transfer device configured to transfer said cuttings sample from said packaging system to a sample storage location.

### BRIEF DESCRIPTION OF THE DRAWINGS

Preferred embodiments of the invention for illustrative use are provided to further clarify the aspects of the invention. Specific reference will be made in the detailed description to the following figures, wherein:

FIG. 1 is an overview of a drilling system;

FIG. 2 shows a preferred drilling mud probe in additional detail;

FIG. 3 shows the preferred drilling mud probe of FIG. 2 in additional detail;

FIG. 4 is a schematic showing a layout of elements arranged in a mud-logging cabin;

FIG. 5 shows a schematic isometric layout of exemplary elements contained in a mud-logging cabin;

FIG. 6 shows a schematic layout of a preferred embodiment of elements in a mud-logging cabin.

FIG. 7 is a flowchart representing steps performed by the system.

The figures are not drawn to scale but serve as supplemental illustrations to further clarify the detailed description.

### DETAILED DESCRIPTION OF PREFERRED EMBODIMENTS

In the following description, specific terms are used to denote known and understood aspects in the art.

Notably, “drilling mud”, as used herein represents drilling fluids of various base solvents, solutes to improve drilling processes, and the material emanating from the drilling process. The drilling mud is a suspension which, in addition to the oil or water-based drilling fluid, includes a number of cuttings, cavings, and/or gas/oil from the rock being perforated by a well drilling procedure when returning from the well head. In this context then, drilling mud is understood to additionally contain cuttings from the drilling operation.

The term “cuttings sample” is understood in the context of the following description as the plurality of rock cuttings extracted from a mud circuit. The term sample is added to denote specific groups of cuttings are related to the interval over which said cuttings are collected. Additional terms may be added to differentiate the cuttings samples between process steps but are related to the same cuttings samples.

Reference 100 denotes a mud circuit employed by the drilling operation. At the bottom of the well, drill bit 130 performs the drilling operation.

Mud circuit 100 comprises a mud return system 111 which receives the drilling mud exiting the head of the well. The mud return system 111 comprises a mud flow line 110. The mud return system 111 optionally comprises a header box 141 located between the mud flow line 110 and a shale shaker 140. The header box 141 is optionally mounted on the shale shaker 140 and is configured to distribute the drilling mud across the shale shaker 140.

As said, mud circuit 100 further comprises one or more shale shakers 140, arranged downstream the mud flow line 110, in particular downstream the header box 141, and adapted to receive the drilling mud flowing in the mud flow line 110, possibly through the header box 141.

Mud circuit 100 further comprises one or more mud reservoirs 145, wherein the drilling mud filtered by shale shaker(s) 140 is stored.

Mud circuit 100 further comprises one or more mud pumps 150 which, when necessary, draw(s) the filtered drilling mud stored in the reservoir 145 and pump(s) it back into the well.

Mud circuit 100 further comprises a secondary mud flow line 160, which departs from the mud flow line 110 and causes part of the drilling mud to flow to a variable flow mud pump, which will be further disclosed in the following.

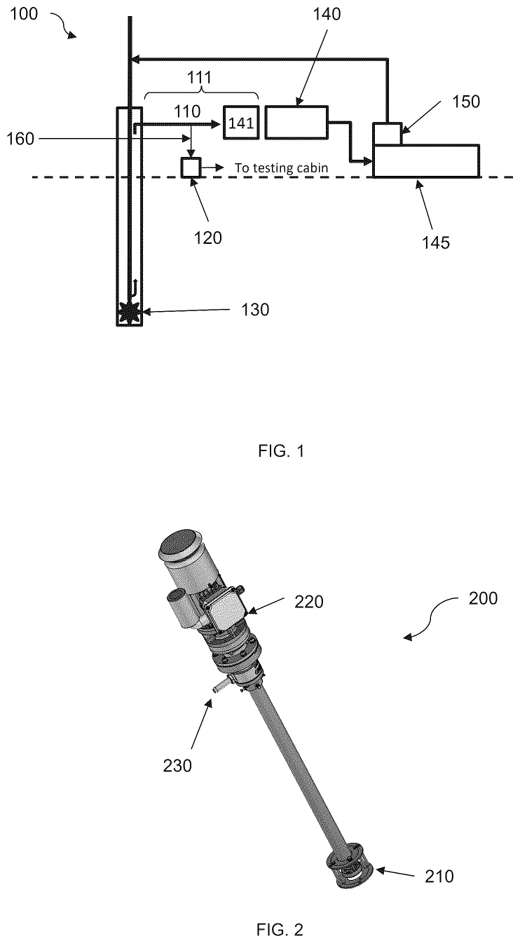

Thus, the mud flow line 110 in the mud circuit 100 shown in FIG. 1 contains drilling mud returning from the well.

The automation of a subsequent mud-logging procedure by means of an intelligent cuttings sampler begins by siphoning off a drilling mud from the mud circuit 100 of the drilling operation.

The siphoning of a certain amount of drilling mud occurs via a mud flow line pass-through which allows a probe 200 to be placed along the mud return system 111.

In the specific use cases of drilling muds, the presence of cavings, i.e., rock pieces coming from geological formations along the well bore at different locations away from the drill bit, can bias or degrade the quality of a subsequent analysis. In practice, cavings are characterized by a larger dimension than cuttings. The difference in dimension between cuttings and cavings enables an initial mechanical selection of rock material contained inside a drilling mud.

The initial mechanical selection of rock is performed by the probe 200, an embodiment of which is shown in FIG. 2. The probe 200 comprises a probe body 210, a connection to a secondary mud flow line 160, and may optionally comprise a cleaning system 220.

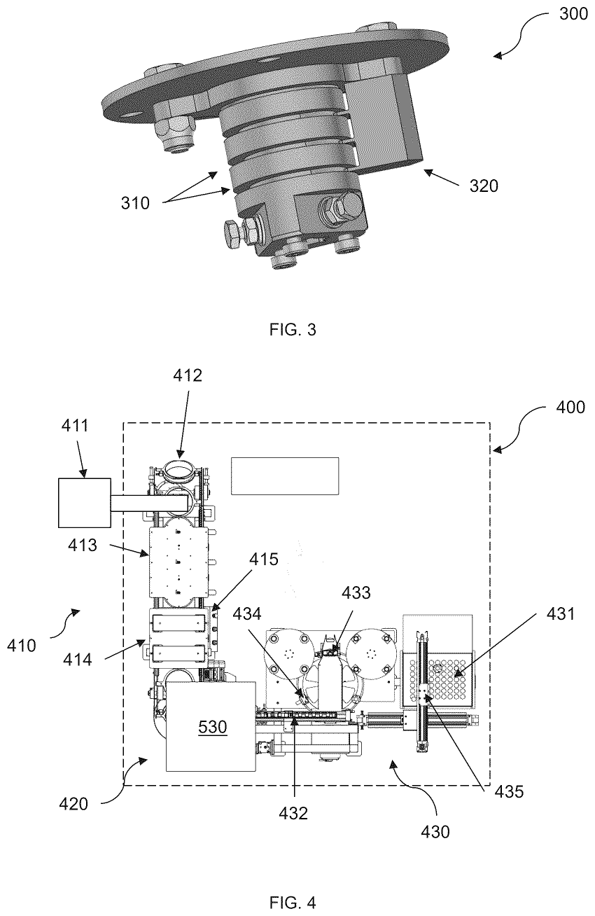

Preferably, probe body 210 has internal features that are shown in detail in FIG. 3. Contained within the probe body 210 are a plurality of openings 310 which allow the drilling mud to pass from the mud flow line 110 into a secondary mud flow line 160 with a secondary mud line connection 230. The probe openings 310 are sized to allow for the selective passage of the cuttings suspended in the drilling mud.

According to an exemplary embodiment, the probe opening is between 0.125 mm and 5 mm in size. Additional opening distances are allowable depending on specific data from a well site. An embodiment of the probe 200 additionally allows for a single opening to allow for the sampling of the drilling mud.

The cleaning system 220 comprises a motor and optionally a gearbox which are connected to the probe body 210. The cleaning system enables the probe body 210 to maintain the probe opening in the mud return system 111 and is further described by the self-cleaning aspect below.

The nature of mud flows, more specifically those containing cavings having larger dimensions than the openings in the probe 200, can create blockages preventing the sampling from occurring. The probe 200 is optionally configured to self-clean the plurality of openings 310 to maintain a flow of the drilling mud. In a particular embodiment, this self-cleaning aspect is performed by a cleaner 320, such as for example, a series of cleaning brushes, schematically shown in FIG. 3.

In a preferred embodiment, a probe that can be used in the intelligent cuttings sampler is disclosed in U.S. patent application Ser. No. 18/526,174 (US Patent Publication No. 2025/0122796 A1), herein incorporated by reference.

The probe 200 may be continuously cleaned or cleaned according to a control signal. The control signal may be provided according to a sensor detecting a flow blockage; the sensor may be one of a flow sensor, mud vacuum pressure sensor, or the like, capable of detecting transients in the siphoned secondary mud flow line 160. The sensor may be mounted inside the secondary mud flow line 160, or any similar location capable of sensing the characteristics of the flow. In this way, the self-cleaning action may be performed only when necessary to maintain the desired siphoned amount.

The probe 200 connects the mud flow line 110 to the secondary mud flow line 160 to pass the drilling mud to subsequent stations belonging to the system.

In connecting the mud flow line 110 to the rest of the system, a variable flow mud pump 120 is connected along the secondary mud flow line 160. The variable flow mud pump 120 draws in the drilling mud from the mud return system 111 through the probe 200, taking a fraction of the total flow for analysis. The amount is typically between 2-5% of the total drilling mud flow. The percentage of mud flow extracted by the variable flow mud pump 120 is variable, able to provide a range of flow rates.

For each interval selected for sampling, the cuttings sample contained in the flow extracted by the variable flow mud pump 120 is representative of the entire interval. Representative cuttings samples contain cuttings extracted at substantially the same percentage over the entire interval. This creates a cuttings sample composed of cuttings equally spaced along the interval being sampled as opposed to overrepresentation of the beginning, middle, or end of the interval.

The variable flow mud pump 120 enables additional sample collection. The variable flow mud pump 120 allows drilling mud samples to be collected from the flow line over defined intervals by controlling the fraction extracted from the mud flow line 110. The defined interval may be computed based on a desired or required amount of cuttings for analysis. This computation may be made and controlled as known in the art according to sample lagging calculations as well as mud flow and drilling rates to create composite samples. The resulting cuttings sample may therefore contain an acceptable amount of cuttings. An acceptable amount of cuttings may be defined by the resulting analyses to be performed on the cuttings or defined by a user. Typically, this acceptable amount is in the range of 100 to 200 grams, with the potential to extend this value if further and/or additional destructive tests will be performed.

Sample collection may be performed at intervals too quickly for manual sampling at the shale shaker 140. The current sampling frequency in practice is on the order of 3 meters. Depending on the drilling rate, collecting samples at that sampling frequency can be very difficult or impossible for manual collection.

Of particular importance is the ability to perform so-called “spot” samples. In contrast to composite sampling, which collects cuttings over an interval distance on the order of 3 meters, “spot” sampling decreases this interval distance. In these instances, the variable flow mud pump 120 may be commanded to operate at a maximum suction to enable fine sampling at specific locations of interest. The variable flow mud pump 120 enables this procedure through its variable pump rate which can increase the drilling mud sample rate at specific times from the typical sample rate to a rate that much exceeds standard collection procedures. In practice, this spot may be on the order of 0.5-1 meters.

The variable flow mud pump control signal is provided by a main controller. The main controller is configured to send control signals to the variable flow mud pump 120. The control signal is dependent upon the acceptable amount of cuttings desired for a sampling activity. Preferably, this control signal is continuously calculated based on changes in the expected ratio of cuttings in the drilling mud dependent upon the drilling parameters.

The acceptable amount of cuttings may be determined as a function of the rate of penetration, interval selection, mud circuit flow rate, the dimension of the drill bit, and/or other similar parameters as known in the art. The determination of an acceptable amount of cuttings returns a desired amount of drilling mud to collect over the chosen interval. An additional buffer amount may be designated as an additional parameter for determining the control signal which controls the variable flow mud pump 120.

The function may be advantageously chosen based on a desire to perform sampling over specific intervals, over varying intervals, or at a specific location using the aforementioned spot sample collection.

Advantageously, an operator may choose to perform an array or combination of these sample collections. An exemplary embodiment of the invention enables the main controller to predefine locations along the depth of a well where to perform specific spot sampling in addition to defining a general sampling frequency over the rest of the drilling operation. The spot sampling may be defined prior to the beginning of the drilling operation or selected by an operator while in the midst of the drilling operation or any combination thereof.

FIG. 4 represents a particular embodiment which contains further stations and/or processing steps of the mud logging procedure.

Passing through the secondary mud flow line 160, the extracted drilling mud arrives at a cleaning system 410. An initial stage of the cleaning system 410 comprises an initial shale shaker 411. The initial shale shaker 411 accepts the drilling mud from the secondary mud flow line 160 and separates the majority of the mud from the cuttings sample. The initial shale shaker 411 is a smaller secondary unit which is capable of handling the flow rate siphoned by the sampling system 200. The initial shale shaker 411 may be any kind of device which removes excess fluid from the solid cuttings contained inside the drilling mud. The initial removal of the drilling mud from the cuttings by the shale shaker may optionally be performed by any similar device known in the art.

The initial shale shaker 411 may be located inside or outside of a testing cabin 400. The testing cabin 400 may contain a number of operators which perform a number of operations on a well-site.

The possibility of toxic gases trapped in the drilling mud and escaping into the working environment may pose a potential health risk to nearby operators. Toxic gases may be contained in and released from the drilling mud when exposed via the initial shale shaker 411 or during the subsequent cleaning process. Potential toxic gases may include gases such as H2S, CO2, SO2, and the LEL of combustible gases contained in the drilling mud. To mitigate risks to nearby operators, a fume hood may be located above the operating area to cover the processing of the cuttings sample when performed inside the testing cabin 400. The fume hood is adapted to protect nearby operators from toxic gases until the cuttings sample is completely dried after the cleaning system 220.

The fume hood permits the initial shale shaker 411 to be optionally located inside of the testing cabin 400 without posing a danger to operators in the vicinity. In addition to the fume hood, a gas sensor may be provided to determine the quantity and/or concentration of toxic gases potentially escaping from the fume hood into the testing cabin 400. Additional gas sensors may be provided for additional protection as necessary for the system to protect operators.

In a particular embodiment of the invention shown in FIG. 6, the cuttings sample from the initial shale shaker 411 is optionally passed to a collection jar. The collection jar may be sized to contain a plurality of cuttings samples. The collection jar preferably contains a quantity of cuttings equivalent to six individual cuttings samples. In practice, this may be equivalent to 1.5 to 2 liters of cuttings.

The collection jar may distribute the cuttings from the initial shale shaker 411 along 3 different paths; waste, washed, and/or unwashed samples. The waste path ends with the initial shale shaker 411 where the cuttings non selected for conservation are immediately discarded as waste. The washed samples are collected and moved to further cleaning stages. The unwashed samples are collected but removed before further cleaning may occur.

The unwashed samples are moved to sample jars. The sample jars may hold a smaller subset of the cuttings in the collection jar, preferably around 500 mL. After the unwashed samples are stored in the sample jars, a bactericide may be added. The unwashed sample is then sealed shut to prevent the fermentation of the organic matter contained in the mud or potential hydrocarbons in the sample. The sample jar can be closed and sealed by pressing or otherwise fixing a lid to the sample jar. After being closed and/or sealed, the sample jar may have a label printed and affixed with information related to the cuttings sample such as time, location, depth, or other similar values. The sample jar is then placed in a storage box specific to the unwashed samples.

After removing the majority of the drilling mud from the cuttings, the partially cleaned cuttings are moved from the initial shale shaker 411 to additional cleaning stages to remove the remaining mud from the cuttings for further analysis.

The cleaning system 410 then passes the cuttings from the initial shale shaker 411 to further cleaning steps by placing the cuttings in a container attached to a first conveyor belt 412.

The first conveyor belt 412 is configured to bring the container from the initial shale shaker 411 to a washing station 413 and a drying station 414. The container may be preferably made of mesh or other porous materials to allow for the drainage of fluid when run under a washing station.

According to an embodiment, the washing station 413 is configured to wash the remaining drilling mud from the cuttings using a cleaning agent.

The cleaning agent may be one of water, recycled water, detergent, solvent and/or other cleaning product as required depending upon the drilling mud components/ingredients.

In some cases, oil-based muds may be washed with diesel or other specialized cleaning agents. The cleaning may occur by rotating nozzles which spray the cuttings with the cleaning agent from multiple directions or other substantially similar method of cleaning. In preferred embodiments of the invention, the washing station 413 performs a multi-step washing operation consisting of a first, second and third washing action.

For water-based muds, the process begins with a first washing action or pre-wash using water alone to remove the majority of leftover mud and prevent clogging of the washing system, followed by a second washing action with detergent where high-pressure nozzles apply the cleaning agent to thoroughly remove drilling mud and residual oil from the cuttings. Finally, a third washing action with water ensures that all traces of mud and detergent are removed, leaving the cuttings ready for downstream processing or disposal.

For oil-based muds, the process begins with a first washing action with solvent (such as diesel) to dissolve and soften the sticky residues, followed by second washing action using a detergent where the cleaning agent is applied to breakdown and remove the remaining mud and oil. The process concludes with a third washing action using water to ensure all contaminants are eliminated.

Preferably, the drying station 414 comprises a heating element and/or a shaker element. The drying station 414 accepts the cuttings after a washing operation is performed. The container of cuttings is passed via the first conveyor belt 412 from the washing station 413 to the drying station 414. The container is enclosed by the drying station 414 and the cuttings are heated and/or vibrated by the respective elements until dried.

In a particular embodiment, after a washing operation has been performed, the container which contains the washed cuttings is moved along the conveyor belt to an imaging system 415. The imaging system 415 may be located between the washing station 413 and the drying station 414 or after the drying station 414.

The imaging system 415 is configured to evaluate the quality and/or completeness of the washing stage. According to a particularly advantageous embodiment, the imaging system 415 can signal to the main controller to re-perform a washing step if a first wash is not adequate to remove the drilling mud from the cuttings.

The cleanliness analysis performed by the imaging system 415 can be performed by a computer vision system or any similar technology. The cleanliness analysis may be performed by a pre-trained neural network, for example using supervised deep learning classification models to make qualitative determinations on cleanliness.

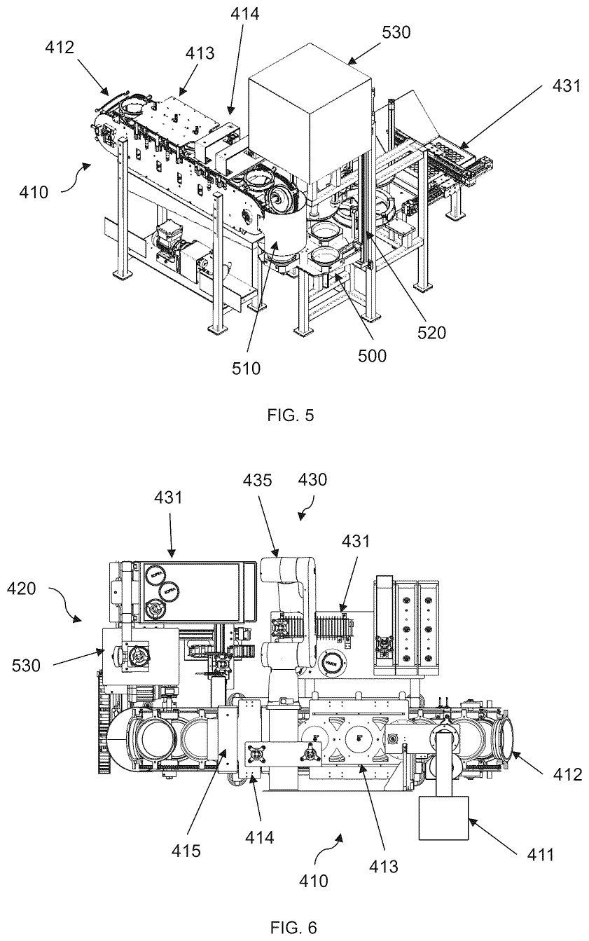

As shown in FIG. 5, after the successful cleaning of the container of cuttings, the container on the first conveyor belt 412 is preferably deposited onto a transport device 500, such as for example a rotating table. The deposit may occur using a funnel 510 or the like. In a particular embodiment, the transport device 500 is provided to minimize the linear dimension of the entire system. Preferably, the transport device 500 is configured to rotate, thus moving the container located on the transport device 500 to various positions for further processing and analysis.

In an embodiment, one location of the transport device 500 corresponds to an analysis system 420. Preferably, the analysis system 420 is comprised of a photobox 530, an image sensor, and a lighting system.

The photobox 530 is configured to block all external light from around the container on the transport device 500 and contain the photographic elements of the analysis system 420. In a particular embodiment, the photobox 530 is configured to slide on a vertical rail 520 disposed above the transport device 500. At a bottom location along the vertical rail 520, the photobox 530 covers the container containing cuttings and completely blocks external sources of light from entering.

The image sensor is a digital image sensor. In a particular embodiment, the image sensor may be one taken from a commercially available digital camera and may be capable of detecting visible light. Optionally, the image sensor may be additionally capable of detecting light over a larger range of electromagnetic radiation, i.e a hyperspectral or multispectral image sensor. Preferably the image sensor may detect ultraviolet light and/or infrared light as well. The image sensor may optionally include multiple image sensors, each adapted to different ranges of the electromagnetic spectrum.

The lighting system is provided to illuminate the cuttings inside the photobox 530. The lighting system comprises white lights to provide a controlled amount of illumination to the cuttings. Additionally, the lighting system comprises ultraviolet (uv) lights. The standard amount of light enables the analysis system 420 to normalize photos of the cutting sample against past and future samples to obtain an image-based analysis of the cuttings.

In embodiments of the invention, the analysis system 420 may further comprise an X-Ray Fluorescence, XRF, module, an X-Ray Diffraction, XRD, module, a Laser Induced Breakdown Spectroscopy, LIBS, module, and/or any combination thereof. The XRF module shoots X-rays on a portion of the sample and determines the elemental composition of the cuttings sample after the photography.

The XRD module focuses X-rays on a small portion of the sample and detects the angle at which X-rays are diffracted to determine the mineralogical composition of the cuttings sample after the photography.

The LIBS module uses laser impulses to bring a small portion of the sample surface to plasma status and detect the elemental composition of the sample.

The analysis system 420 may be additionally responsible for determining cuttings sample characteristics based on the results of the various sensor modules. The analysis system 420 may be configured to determine at least a size and shape of the cutting fragments which may be identified in the cuttings sample by images taken from the image sensor. Additional statistics related to the deviation and spread of cutting sizes and shapes may also be determined. Using the size, shape, and their associated statistical properties to determine qualitative and quantitative assessments for optimal drilling mud properties. The analysis system 420 may be tasked to compute mud weight and/or viscosity to enhance cutting transport. The analysis system 420 may be utilized to recognize if sample mixing is happening and the sample is not representative of a single depth interval.

In an embodiment, one location of the transport device 500 corresponds to a packaging system 430. The packaging system 430 is provided to automatically store the different cuttings samples for various logging purposes. The packaging system 430 comprises a sample vessel stock device 431, a cuttings transfer device 432, a capping device 433, a labeling device 434, and a vessel transfer device 435.

The sample vessel stock device 431 provides empty or evacuated sample vessels for each sample being extracted from the drilling mud flow. This device may optionally take the form of a vertical vessel storage device or other similar storage device for sample vessels. The vessel stock device 431 provides a plurality of sample vessels to the packaging system.

The cuttings transfer device 432 includes a vibrational channel which accepts the cuttings sample from the transport device 500 and deposits them into a sample vessel provided by the sample vessel stock device 431. The deposit may utilize a funnel system or other method of depositing the cuttings sample into the sample vessel.

Optionally, the cuttings transfer device 432 may include a sensor which is configured to measure the amount of cuttings contained in the sample vessel. The measurement determines an overfilling quantity to enable a control on the quantity of cuttings in the samples as they are placed into storage.

The sample vessel containing the cuttings sample is then covered by the capping device 433 and labeled by the labeling device 434 before being passed onto the vessel transfer device 435. Vessel transfer device 435 is configured to retrieve the sample vessel from the labeling device 434 and deposit the sample vessel in a sample storage location, such as a sample storage container.

The vessel transfer device 435 can take the form of any number of physical structures which transfers or is capable of transferring a sample vessel to a sample storage location. In a particular embodiment, the vessel transfer device 435 is a robotic arm. Alternatively, and for simplicity, the vessel transfer device 435 may comprise a multi-axis/dual-axis system which retrieves and deposits the sample.

Preferably, the sample vessel provided by the sample vessel stock device 431 is transferred between the cutting transfer device 432, the capping device 433, the labeling device 434, and the vessel transfer device 435 by means of a carousel.

In a particular embodiment of the invention, the system is additionally equipped with a plurality of automatic cleaning stations. The automatic cleaning stations provide clean water jets to sanitize and clean various sections of the system.

An automatic cleaning station may be located before the sieve loading stage for the cuttings sample.

An automatic cleaning station may be located below the first conveyor belt 412, after the drying station 414, and between the first conveyor belt 412 and the transport device 500.

An automatic cleaning station may provide cleaning inside the collection jar after every sample distribution. The cleaning process prevents cross contamination between sample collections.

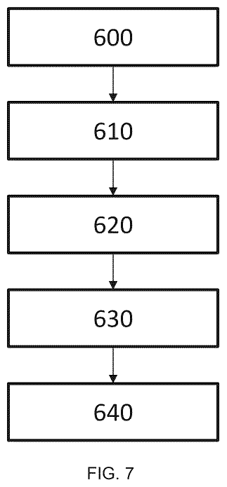

FIG. 7 shows a processing flowchart for the completion of a mud logging procedure performed by the system. Beginning from a first step 600 of sampling a percentage of the mud flow from the well head, the system is able to obtain a number of cuttings which represent specific locations or intervals along the bore hole.

Step 610 concerns the transfer of the obtained cuttings between the mud sampling and further processing. The obtained cuttings from the initial shale shaker 411 may be optionally placed into a collection jar to help facilitate the transfer of the cuttings. The collection jar is configured to perform a cuttings sample distribution among the three paths. The unwashed samples and waste samples can be selectively diverted from the washed sample path which requires additional cleaning and either discarded directly and/or stored unwashed in the sample jar.

Step 620 is the cleaning step. The extracted cuttings are still contained in the slurry when sampled from the mud flow line 110. In order to perform the analyses, the cuttings are preferably clean and free from other debris. The cleaning step is performed by the cleaning system 410 as mentioned and described in detail above. In cases where the cleaning system 410 may require additional cleaning, the cleaning system 410 may be configured to perform heterogeneous cleaning stages depending upon a mud type. For a water-based mud, a typical cleaning may comprise a water, detergent, and subsequent water stage. For an oil-based mud, a typical cleaning may comprise a solvent, detergent, and water stage, where the solvent is typically diesel fuel.

Step 630 is the analysis step. The cleaned and dried cuttings, having been moved from the cleaning system 410, are moved to the analysis system 420. The analysis system 420 is configured to perform various visual inspections on the cuttings sample. The results of the analysis are noted for the associated drilling depth and logged for reporting to the well operator.

Step 640 is the packaging step. After analysis, the cuttings are then moved to storage by means of the packaging system 430. The packaging step encompasses transferring the cuttings into a vessel, capping the vessel, and affixing a label to the vessel marking the cuttings sample with a unique id to associate the same cuttings sample with a specific analysis result and bore location.

The intelligent cuttings sampler then contains all aspects and equipment necessary to complete a mud-logging procedure without operator intervention.

1. An intelligent cuttings sampler for the retrieval and testing of cuttings from a drilling operation comprising:
a sample extractor which extracts a sample of drilling mud from a mud return system, comprising:
a probe, wherein the probe is located inside of the mud return system and has a plurality of openings sized to allow for the passage of drilling mud containing cuttings, and
a variable flow mud pump for extracting a drilling mud sample from the mud return system,
a cleaning system which is configured to accept the drilling mud sample provided by the sample extractor and performs a cleaning action to remove fluid in the drilling mud sample resulting in a cutting sample;
an analysis system for performing an initial analysis on the cutting sample to classify a type of rock contained in the cutting sample,
a packaging system for accepting and placing the cutting sample in a storage container, and
a main controller, configured to control at least the variable flow mud pump as a function of a rate of penetration of the drilling operation.

2. The intelligent cuttings sampler of claim 1, wherein said function of a rate of penetration is based on an estimate of an acceptable amount of cuttings contained in a drilling mud sample.

3. The intelligent cuttings sampler of claim 1, further comprising wherein said probe includes a cleaner capable of maintaining said plurality of openings free of obstructions.

4. The intelligent cuttings sampler of claim 3, wherein said plurality of openings have a dimension of between 3 mm and 4 mm.

5. The intelligent cuttings sampler of claim 1, wherein said cleaning system further comprises an initial shale-shaker configured to accept said drilling mud sample from said sample extractor and separate cuttings from drilling mud.

6. The intelligent cuttings sampler of claim 5, further comprising:
a gas sensor configured to send information pertaining to a quantity or concentration of a hazardous gas,
wherein at the initial shale-shaker the quantity or concentration of a hazardous gas is measured by said gas sensor inside an associated fume hood.

7. The intelligent cuttings sampler of claim 1, wherein the cleaning system further comprises:
a washing station configured to perform a first washing action being performed with a solvent selected based on a type of the drilling mud, a second washing action being performed with detergent, and a third washing action being performed with water,
a drying station configured to perform a drying operation using a heating element and/or a vibration element.

8. The intelligent cuttings sampler according to claim 6, wherein said cleaning system further comprises an imaging system located in between said washing and drying station and wherein said imaging system is configured to analyze a cleanliness level of said cutting sample after said first and/or said second washing action.

9. The intelligent cuttings sampler of claim 1, wherein the analysis system comprises a photography device capable of taking photographs in ultra-violet and visible light and wherein the classification of rock contained in the cutting sample is based on the output of a trained machine learning algorithm.

10. The intelligent cuttings sampler of claim 9, wherein the photography device is a hyperspectral camera.

11. The intelligent cuttings sampler of claim 1, wherein the packaging system is configured to separate a subset of said cutting sample for additional analysis.

12. The intelligent cuttings sampler of claim 1, further comprising:
a first conveyor belt with a plurality of mesh containers configured to accept said drilling mud sample and enable said cleaning system (410) to perform said cleaning action;
a transport device containing a plurality of sample containers configured to transfer said cuttings sample between said cleaning system, analysis system and packaging system, and allow for compact layout of said cleaning, analysis, and packaging systems.

13. The intelligent cuttings sampler according to claim 11, further comprising a vessel transfer device configured to transfer said cuttings sample from said packaging system to a sample storage location.

14. The intelligent cuttings sampler according to claim 5, further comprising a collection jar configured to receive cuttings samples from the initial shale shaker and distribute them along 3 different paths; unwashed, washed, and/or waste.

15. The intelligent cuttings sampler according to claim 14, wherein unwashed samples are moved to sample jars with a bactericide and sealed and packed for storage.

16. The intelligent cuttings sampler according to claim 1, wherein said analysis system further comprises at least one of a XRD module, XRF module, and/or a LIBS module.
